SEQUENCE LISTING (13 sequences)

SEQ ID NO: 1 - protein, 120 aa, Artificial Sequence
  Synthetic peptide sequence
QVQLVQSGAEVKKPGASVKVSCKASGYTFTGYYIHWVRQAPGQGLEWMGWIYPGNFNTKYNERFKGRVTMTTDTSTSTAYMELRSLRSDDTAVYYCAREDGSPYYAMDYWGQGTSVTVSS

SEQ ID NO: 2 - protein, 5 aa, Artificial Sequence
  Synthetic peptide sequence
GYYIH

SEQ ID NO: 3 - protein, 7 aa, Artificial Sequence
  Synthetic peptide sequence
GYTFTGY

SEQ ID NO: 4 - protein, 17 aa, Artificial Sequence
  Synthetic peptide sequence
WIYPGNFNTKYNERFKG

SEQ ID NO: 5 - protein, 6 aa, Artificial Sequence
  Synthetic peptide sequence
YPGNFN

SEQ ID NO: 6 - protein, 11 aa, Artificial Sequence
  Synthetic peptide sequence
EDGSPYYAMDY

SEQ ID NO: 7 - protein, 449 aa, Artificial Sequence
  Synthetic peptide sequence
QVQLVQSGAEVKKPGASVKVSCKASGYTFTGYYIHWVRQAPGQGLEWMGWIYPGNFNTKYNERFKGRVTMTTDTSTSTAYMELRSLRSDDTAVYYCAREDGSPYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 112 aa, Artificial Sequence
  Synthetic peptide sequence
DIVMTQTPLSLSVTPGQPASISCRSSQSLVHSNGNTFLYWYLQKPGQSPQLLIYRVSNRFSGVPDRFSGSGSGTDFTLKISRVEAEDVGVYYCFQATHVPWTFGGGTKVEIK

SEQ ID NO: 9 - protein, 16 aa, Artificial Sequence
  Synthetic peptide sequence
RSSQSLVHSNGNTFLY

SEQ ID NO: 10 - protein, 11 aa, Artificial Sequence
  Synthetic peptide sequence
QSLVHSNGNTF

SEQ ID NO: 11 - protein, 7 aa, Artificial Sequence
  Synthetic peptide sequence
RVSNRFS

SEQ ID NO: 12 - protein, 9 aa, Artificial Sequence
  Synthetic peptide sequence
FQATHVPWT

SEQ ID NO: 13 - protein, 219 aa, Artificial Sequence
  Synthetic peptide sequence
DIVMTQTPLSLSVTPGQPASISCRSSQSLVHSNGNTFLYWYLQKPGQSPQLLIYRVSNRFSGVPDRFSGSGSGTDFTLKISRVEAEDVGVYYCFQATHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2018/0099054 A1
(43) Pub. Date: Apr. 12, 2018

(54) TREATMENT WITH ANTI-EFNA4 ANTIBODY-DRUG CONJUGATES

(71) Applicants: PFIZER INC., NEW YORK, NY (US); RINAT NEUROSCIENCE CORP., SOUTH SAN FRANCISCO, CA (US)

(72) Inventors: ASHWIN GOLLERKERI, LEXINGTON, MA (US); KAREN VELASTEGUI, CARLSBAD, CA (US); RAFFAELE BAFFA, WELLESLEY, MA (US); JAYMES SCOT HOLLAND, LOS ANGELES, CA (US)

(73) Assignees: PFIZER INC., NEW YORK, NY (US); RINAT NEUROSCIENCE CORP., SOUTH SAN FRANCISCO, CA (US)

(21) Appl. No.: 15/573,303

(22) PCT Filed: May 12, 2016
(86) PCT No.: PCT/US2016/032164

§ 371 Date: Nov. 10, 2017

Related U.S. Application Data
(60) Provisional application No. 62/160,861, filed on May 13, 2015.
(60) Provisional application No. 62/168,208, filed on May 29, 2015.

(51) Int. Cl. A61K 47/50; C07K 16/28; A61P 35/00; A61K 48/00

(52) U.S. Cl. CPC A61K 47/50; C07K 16/28; A61P 35/00; A61K 2039/505; C07K 2317/53; C07K 2317/92; A61K 48/0083

(57) ABSTRACT

The present invention provides for dosing regimens for the treatment of patients with cancer and/or an EFNA4-associated disorder with an anti-EFNA4 antibody-drug conjugate (ADC). The present invention further provides for methods for the treatment of patients with cancer and/or an EFNA4-associated disorder in which an anti-EFNA4 ADC is administered intravenously weekly (QW) or every 3 weeks (Q3W).

### RELATED APPLICATIONS

Priority is claimed to provisional U.S. Application No. 62/168,208 filed May 29, 2015 and provisional U.S. Application No. 62/160,861 filed May 13, 2015, which are incorporated by reference herein in their entirety.

### SEQUENCE LISTING

This application is being filed electronically via EFS-Web and includes an electronically submitted sequence listing in .txt format. The .txt file contains a sequence listing entitled “PC72228_Sequence_Listing.txt” created on May 10, 2016, and having a size of 10 KB. The sequence listing contained in this .txt file is part of the specification and which is incorporated herein by reference in its entirety.

### FIELD OF THE INVENTION

The present invention relates to therapeutic regimens for treatment of patients with cancer and/or ephrin-A4 ligand (EFNA4) associated disorders. The subject therapeutic regimens involve administration of an anti-EFNA4 antibody-drug conjugate (ADC) to patients in need thereof.

### BACKGROUND

Ephrin receptors (EPH), the largest family of receptor tyrosine kinases, are type-I transmembrane proteins that bind ephrin ligands (EFN). Receptors in the EPH subfamily typically have a single kinase domain and an extracellular region containing a Cys-rich domain and 2 fibronectin type III repeats. Based upon sequence analyses, ephrin ligands can be divided into two groups: six ephrin-A ligands (EFNA) and three ephrin-B ligands (EFNB). EFNA ligands (i.e., EFNA1, EFNA2, EFNA3, EFNA4, EFNA5, EFNA6) are typically anchored to the cell surface via glycosyl phosphatidylinositol (GPI) linkages, although some non-GPI-anchored proteins are produced through alternative splicing of ephrin mRNAs, such as EFNA4. EFNB ligands (i.e. EFNB1, EFNB2, EFNB3) contain a transmembrane domain and a short cytoplasmic region with conserved tyrosine residues and a PDZ-binding motif. EFNA ligands preferentially bind with any of the nine different ephrin A receptors (EPHA) (i.e., EPHA1 , EPHA2, EPHA3, EPHA4, EPHA5, EPHA6, EPHA7, EPHA8, EPHA9), whereas EFNB ligands preferentially bind with any of six different ephrin B receptors (EPHB) (i.e. EPHB 1, EPHB2, EPHB 3, EPHB4, EPHB 5, EPHB 6), although some cross-interactions have been reported.

EFN-EPH signaling can be bi-directional (impacting both the ligand- and receptor-expressing cells) and regulates a broad range of biological activities including neural development, cell patterning, angiogenesis, and cell motility and invasion. In the context of cancer, the expression of various EPHs and EFNs has been observed, and various functions have been reported (Hafner et al., Clinical Chemistry 50(3):490-499, 2004; Surawska et al., Cytokine & Growth Factor Reviews 15(6):419-433, 2004; Pasquale, E. B, Nature Reviews Cancer 10(3):165-180, 2010). Due to ligand-receptor binding promiscuity as well as functional overlap, it has been difficult to precisely define the roles of each EFN and EPH. While therapeutic targeting of the EPH receptors for the treatment of cancer has been explored, targeting of EFN ligands has not been pursued to any great extent (Pasquale, E. B., Nature Reviews Cancer 10(3):165-180, 2010). There remains a significant need for therapies that provide clinical benefit for the treatment of patients with cancer and/or EFNA4-associated disorders.

### SUMMARY

The present invention provides dosing regimens for the treatment or prophylaxis of cancer and/or an EFNA4-associated disorder with an anti-EFNA4 antibody-drug conjugate (ADC). In some aspects of the invention, a dosage regimen comprises administering an effective amount of an anti-EFNA4 antibody-drug conjugate to a patient at least twice every week, at least weekly (QW), at least every 2 weeks (Q2W), at least every 3 weeks (Q3W) or at least every 4 weeks (Q4W). In particular aspects of the invention, a dosage regimen comprises administering an effective amount of an anti-EFNA4 antibody-drug conjugate to a patient weekly (QW) or every 3 weeks (Q3W).

The present invention also provides methods for the treatment or prophylaxis of cancer and/or an EFNA4-associated disorder, comprising administering to a patient an effective amount of an anti-EFNA4 antibody-drug conjugate. In some aspects of the invention, the method comprises administering to the patient an effective amount an anti-EFNA4 antibody-drug conjugate at least twice every week, at least weekly (QW), at least every 2 weeks (Q2W), at least every 3 weeks (Q3W) or at least every 4 weeks (Q4W). In particular aspects of the invention, the method comprises administering to the patient an effective amount of an anti-EFNA4 antibody-drug conjugate (ADC) weekly (QW) or every 3 weeks (Q3W).

The present invention also provides anti-EFNA4 antibody-drug conjugates for use in the treatment or prophylaxis of cancer and/or EFNA4-associated disorders. The present invention also provides uses of an anti-EFNA4 antibody-drug conjugate in the treatment or prophylaxis of cancer and/or an EFNA4-associated disorder. The present invention also provides uses of an anti-EFNA4 antibody-drug conjugate in the manufacture of a medicament for treatment or prophylaxis of cancer and/or an EFNA4-associated disorder. The present invention also provides pharmaceutical compositions comprising an anti-EFNA4 antibody-drug conjugate for use in the treatment or prophylaxis of a cancer and/or an EFNA4-associated disorder.

In some aspects of the invention, administration of, or use of, a pharmaceutical composition or formulation comprising an anti-EFNA4 antibody-drug conjugate is contemplated.

The present invention also provides anti-EFNA4 antibody-drug conjugates formulated as a pharmaceutical composition. The present invention also provides methods of preparing and manufacturing anti-EFNA4 antibody-drug conjugates and pharmaceutical compositions comprising the same. The present invention also provides article of manufacture and kits comprising the pharmaceutical compositions disclosed herein.

In some aspects of the invention, the anti-EFNA4 antibody-drug conjugate is administered or is administrable at a dose of about 0.010 mg/kg to about 10 mg/kg or any range of dosages between these values. In another aspect of the invention, the anti-EFNA4 antibody-drug conjugate is administered or is administrable at a dose of about 0.010 mg/kg to about 5 mg/kg, about 0.010 mg/kg to about 1 mg/kg, or about 0.010 mg/kg to about 0.500 mg/kg. In some aspects of the invention, the anti-EFNA4 antibody-drug conjugates is administered or is administrable at a dose of at least 0.010, 0.015, 0.020, 0.025, 0.030, 0.035, 0.040, 0.045, 0.050, 0.055, 0.060, 0.065, 0.070, 0.075, 0.080, 0.095, 0.100, 0.150, 0.200, 0.250, 0.300, 0.350, 0.400, 0.450 or 0.500 mg/kg. In some aspects of the invention, dosages of about 0.010 mg/kg, 0.015 mg/kg, 0.020 mg/kg, 0.030 mg/kg, 0.050 mg/kg, 0.075 mg/kg, 0.100 mg/kg, or 0.134 mg/kg are particularly contemplated. In a particular aspect of the invention, the anti-EFNA4 antibody-drug conjugate is administered or is administrable weekly (QW) at a dose of about 0.010 mg/kg, 0.015 mg/kg or 0.020 mg/kg. In another aspect of the invention, the anti-EFNA4 antibody-drug conjugate is administered or is administrable every 3 weeks (Q3W) at a dose of about 0.015 mg/kg, 0.030 mg/kg, 0.050 mg/kg, 0.075 mg/kg, 0.100 mg/kg, or 0.134 mg/kg.

In some aspects of the invention, the anti-EFNA4 antibody-drug conjugates of the present invention comprise an antibody comprising three CDRs from a heavy chain variable region (VH) having the amino acid sequence shown in SEQ ID NO: 1 and three CDRs from a light chain variable region (VL) having the amino acid sequence shown in SEQ ID NO: 8. In another aspect of the invention, anti-EFNA4 antibody-drug conjugates comprise an antibody comprising a VH CDR1 having the amino acid sequence shown in SEQ ID NO: 2, VH CDR2 having the amino acid sequence shown in SEQ ID NO: 4, and VH CDR3 having the amino acid sequence shown in SEQ ID NO: 6, and/or VL CDR1 having the amino acid sequence shown in SEQ ID NO: 9, VL CDR2 having the amino acid sequence shown in SEQ ID NO: 11, and VL CDR3 having the amino acid sequence shown in SEQ ID NO: 12. In some aspects of the invention, the anti-EFNA4 antibody-drug conjugates comprise an antibody comprising a heavy chain variable region (VH) having the amino acid sequence shown in SEQ ID NO: 1 and a light chain variable region (VL) having the amino acid sequence shown in SEQ ID NO: 8. In a particular aspect of the invention, the anti-EFNA4 antibody-drug conjugate comprises an antibody designated huE22, described in U.S. Patent Publication No. 2015/0125472 and International Patent Application Publication WO 2012/118547, each of which is herein incorporated by reference in its entirety. In another aspect of the invention, the anti-EFNA4 antibody-drug conjugate further comprises a drug and a linker, wherein the drug is calicheamicin or a calicheamicin derivative (CM) and the linker is 4-(4′acetylphenoxy)butanoic acid (AcBut). In a particular aspect, the anti-EFNA4 antibody-drug conjugate is huE22-AcBut-CM.

In some aspects of the invention, the cancer and/or EFNA4-associated disorder is characterized by overexpression of EFNA4. In some aspects of the invention, the cancer or EFNA4-associated disorder is breast cancer, triple-negative breast cancer (TNBC), ovarian cancer, colorectal cancer, liver cancer, hepatocellular carcinoma (HCC), lung cancer non-small cell lung cancer (NSCLC), small cell lung cancer (SCLC), appendiceal cancer, spindle cell sarcoma, pancreatic cancer, cervical adenocarcinoma, peritoneal cancer, metastatic melanoma, renal cancer, neoplasm peritoneum, or a leukemia such as chronic lymphocytic leukemia (CLL).

### BRIEF DESCRIPTION OF THE DRAWINGS

FIG. 1 provides the heavy chain amino acid sequences of humanized anti-EFNA4 antibody huE22 (SEQ ID NOs 1-7).

FIG. 2 provides the light chain amino acid sequences of humanized anti-EFNA4 antibody huE22 (SEQ ID NOs 8-13).

FIG. 3 provides the structure huE22-AcBut-CM, showing the humanized IgG1 antibody (huE22), calicheamicin (DNA damaging) payload with AcBut (hydrazine) linker.

FIG. 4 shows the maximum percentage change in tumor size for target lesions by RECIST in patients treated with huE22-AcBut-CM ADC.

FIG. 5A provides the cycle1 mean antibody-conjugated payload serum concentration (ng/mL) versus time (nominal days post dose) profiles of huE22-AcBut-CM ADC at various dosage (mg/kg) for the Q3W regimen.

FIG. 5B provides the cycle1 mean antibody-conjugated payload serum concentration (ng/mL) versus time (nominal days post dose) profiles of huE22-AcBut-CM ADC at various dosage (mg/kg) for the QW regimen.

FIG. 6A provides the cycle1 mean total antibody serum concentration (ng/mL) versus time (nominal days post dose) profiles of huE22-AcBut-CM ADC at various dosage (mg/kg) for the Q3W regimen.

FIG. 6B provides the cycle1 mean total antibody serum concentration (ng/mL) versus time (nominal days post dose) profiles of huE22-AcBut-CM ADC at various dosage (mg/kg) for the QW regimen.

### DETAILED DESCRIPTION

The present invention provides dosing regimens for the treatment or prophylaxis of cancer and/or an EFNA4-associated disorder with an anti-EFNA4 antibody-drug conjugate (ADC) or a pharmaceutical composition comprising the same. In some aspects of the invention, a dosage regimen may comprise administering an effective amount of an anti-EFNA4 antibody-drug conjugate to a patient at least twice every week, at least weekly (QW), at least every 2 weeks (Q2W), at least every 3 weeks (Q3W) or at least every 4 weeks (Q4W). In particular aspects of the invention, a dosage regimen may comprise administering an effective amount of an anti-EFNA4 antibody-drug conjugate to a patient weekly (QW) or every 3 weeks (Q3W). In particular aspects of the invention, the efficacy of the dosage regimen may be determined by measuring the decrease in tumor size as compared to the tumor size in the patient prior to the initial administration of the anti-EFNA4 antibody-drug conjugate. For example, the tumor may decrease in size by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or up to 100%, or up to a point at which the tumor is no longer detectable. The present invention also provides methods for the treatment or prophylaxis of cancer and/or an EFNA4-associated disorder comprising administering an anti-EFNA4 ADC or pharmaceutical composition comprising the same to a patient. The present invention further provides methods for the treatment or prophylaxis of cancer and/or an EFNA4-associated disorder in which an anti-EFNA4 ADC or pharmaceutical composition comprising the same is intravenously administered to a patient weekly (QW) or every 3 weeks (Q3W).

The present invention also provides anti-EFNA4 ADCs and pharmaceutical compositions comprising the same for use in the treatment or prophylaxis of cancer and/or an EFNA4-associated disorder. The present invention further provides anti-EFNA4 ADCs or pharmaceutical compositions comprising the same for use in the treatment or prophylaxis of cancer and/or an EFNA4-associated disorder in which the anti-EFNA4 ADC or pharmaceutical composition comprising the same is intravenously administrable to a patient weekly (QW) or every 3 weeks (Q3W).

The present invention also provides uses of an anti-EFNA4 ADC or pharmaceutical composition comprising the same for treatment or prophylaxis of cancer and/or an EFNA4-associated disorder. The present invention further provides uses of an anti-EFNA4 ADC or pharmaceutical composition comprising the same for treatment or prophylaxis of cancer and/or an EFNA4-associated disorder in which an anti-EFNA4 ADC or pharmaceutical composition comprising the same is intravenously administrable to a patient weekly (QW) or every 3 weeks (Q3W).

The present invention also provides uses of an anti-EFNA4 ADC in the manufacture of a medicament for treatment or prophylaxis of a cancer and/or an EFNA4-associated disorder.

The present invention also provides pharmaceutical compositions comprising an anti-EFNA4 ADC for use in the treatment or prophylaxis of cancer and/or an EFNA4-associated disorder.

The present invention also provides anti-EFNA4 ADCs and pharmaceutical compositions comprising the same for use in the treatment or prophylaxis of a condition associated with EFNA4 expression in a patient. The conditions associated with EFNA4 expression include, but are not limited to, abnormal EFNA4 expression, altered or aberrant EFNA4 expression, EFNA4 overexpression, and a proliferative disorder (e.g., cancer).

The present invention also provides methods for the treatment or prophylaxis of a condition associated with EFNA4 expression in a patient comprising administering an anti-EFNA4 ADC or pharmaceutical composition comprising the same to the patient.

The present invention also provides uses of an anti-EFNA4 ADC or pharmaceutical composition comprising the same for treatment or prophylaxis of a condition associated with EFNA4 expression in a patient.

The present invention also provides uses of an anti-EFNA4 ADC in the manufacture of a medicament for treatment or prophylaxis of a condition associated with EFNA4 expression in a patient.

The present invention also provides pharmaceutical compositions for use in the treatment or prophylaxis of a condition associated with EFNA4 expression in a patient.

The present invention also provides anti-EFNA4 ADCs and pharmaceutical compositions comprising the same for use in inhibiting growth or progression of an EFNA4-expressing tumor in a patient.

The present invention also provides methods for inhibiting growth or progression of an EFNA4-expressing tumor in a patient comprising administering an anti-EFNA4 ADC or pharmaceutical composition comprising the same to the patient.

The present invention also provides uses of an anti-EFNA4 ADC or pharmaceutical composition comprising the same for inhibiting growth or progression of an EFNA4-expressing tumor in a patient.

The present invention also provides uses of an anti-EFNA4 ADC in the manufacture of a medicament for inhibiting growth or progression of an EFNA4-expressing tumor.

The present invention also provides pharmaceutical compositions comprising an anti-EFNA4 ADC for use in inhibiting growth or progression of an EFNA4-expressing tumor.

The present invention also provides anti-EFNA4 ADCs and pharmaceutical compositions comprising the same for use in inhibiting metastasis of EFNA4-expressing cancer cells in a patient.

The present invention also provides methods for inhibiting metastasis of EFNA4-expressing cancer cells in a patient comprising administering an anti-EFNA4 ADC or pharmaceutical composition comprising the same to the patient.

The present invention also provides uses of an anti-EFNA4 ADC or pharmaceutical composition comprising the same for inhibiting metastasis of EFNA4-expressing cancer cells in a patient.

The present invention also provides uses of an anti-EFNA4 ADC in the manufacture of a medicament for inhibiting metastasis of EFNA4-expressing cancer cells.

The present invention also provides pharmaceutical compositions comprising an anti-EFNA4 ADC for use in inhibiting metastasis of EFNA4-expressing cancer cells.

The present invention also provides anti-EFNA4 ADCs and pharmaceutical compositions comprising the same for use in inducing regression of an EFNA4-expressing tumor in a patient.

The present invention also provides methods for inducing regression of an EFNA4-expressing tumor in a patient comprising administering an anti-EFNA4 ADC or pharmaceutical composition comprising the same to the patient.

The present invention also provides uses of an anti-EFNA4 ADC or pharmaceutical composition comprising the same for inducing regression of an EFNA4-expressing tumor in a patient.

The present invention also provides uses of an anti-EFNA4 ADC in the manufacture of a medicament for inducing regression of an EFNA4-expressing tumor.

The present invention also provides pharmaceutical compositions comprising an anti-EFNA4 ADC for use in inducing regression of an EFNA4-expressing tumor.

The present invention also provides anti-EFNA4 antibody-drug conjugates formulated as a pharmaceutical composition. The present invention also provides methods of preparing and manufacturing anti-EFNA4 antibody-drug conjugates and pharmaceutical compositions comprising the same. The present invention also provides articles of manufacture and kits comprising the pharmaceutical compositions disclosed herein.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Gellis, ed., 1998) Academic Press; Animal Cell Culture (R I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995).

As used herein, the terms “antibody-drug conjugate” or “ADC” are used interchangeably and refer to antibodies, or antigen-binding fragments thereof, including antibody derivatives that bind to EFNA4 and are conjugated to a drug such as a cytotoxic, cytostatic, and/or therapeutic agent, as described herein. For example, a cytotoxic agent can be linked or conjugated to an anti-EFNA4 antibody as described herein for targeted local delivery of the cytotoxic agent to tumors (e.g., EFNA4 expressing tumors). Thus, the antibody-drug conjugates of the present invention comprise an antibody, or antigen-binding fragment thereof, that binds to EFNA4, and a linker-drug moiety. In contrast to the tyrosine kinase inhibitors (TKIs) developed to inhibit Eph receptor signaling, antibody-drug conjugates (ADCs), such as anti-EFNA4 ADCs, can target specific surface molecules and the cells expressing them regardless of their signaling function, as long as the molecules efficiently internalize.

As used herein, the term “EFNA4” includes variants, isoforms, homologs, orthologs and paralogs. EFNA4 is also known in the art as ephrin-A4, ephrin-A4 ligand, EPH-related receptor tyrosine kinase ligand 4, Ligand Of Eph-Related Kinase 4, EFL4, EPLG4 and LERK4. In some aspects of the invention, antibodies and antibody-drug conjugates cross-react with EFNA4 from species other than human, such as EFNA4 of mouse, rat, or primate, as well as different forms of EFNA4 (e.g., glycosylated EFNA4). In other aspects, the antibodies and antibody-drug conjugates may be completely specific for human EFNA4 and may not exhibit species or other types of cross-reactivity. As used herein the term EFNA4 refers to naturally occurring human EFNA4 unless contextually dictated otherwise. Therefore, an “EFNA4 antibody”, “anti-EFNA4 antibody”, “ephrin-A4 antibody” or “ephrin-A4 ligand antibody” or other similar designation, means any antibody (as defined herein) that associates, binds or reacts with the EFNA4 type ligand or isoform, or fragment or derivative thereof. Further, an “EFNA4 antibody-drug conjugate”, “anti-EFNA4 antibody-drug conjugate”, “ephrin-A4 antibody-drug conjugate” or “ephrin-A4 ligand antibody-drug conjugate” means any antibody-drug conjugate or ADC (as defined herein) that associates, binds or reacts with the EFNA4 type ligand or isoform, or fragment or derivative thereof. EFNA4 is overexpressed in tumor versus normal tissue in a number of human tumors.

As used herein, the term “linker” describes the direct or indirect linkage of the antibody to the drug. Attachment of a linker to an antibody can be accomplished in a variety of ways, such as through surface lysines, reductive-coupling to oxidized carbohydrates, and through cysteine residues liberated by reducing interchain disulfide linkages. A variety of ADC linkage systems are known in the art, including hydrazone-, disulfide- and peptide-based linkages. In particular aspects of the invention, the linker of EFNA4 antibody-drug conjugates of the invention includes, but is not limited to, 4-(4′acetylphenoxy)butanoic acid (hereinafter “AcBut”).

As used herein, the terms “drug”, “payload” and “compound” are used interchangeably and refer to any substance having biological or detectable activity, for example, therapeutic agents, detectable labels, binding agents, etc., and prodrugs, which are metabolized to an active agent in vivo. In some aspects of the invention, the drug is calicheamicin or a calicheamicin derivative (CM), including N-acetyl derivatives of calicheamicin, such as N-acetyl-γ-calicheamicin and N-acetyl-γ-calicheamicin dimethyl hydrazide (DMH).

As used herein, the term “linker-drug moiety” refers to the molecule resulting from a drug linked or conjugated to a linker.

As used herein, the terms “binding affinity” or “K_{D}” refers to the equilibrium dissociation constant of a particular antigen-antibody interaction. The K_{D} is the ratio of the rate of dissociation, also called the “off-rate” or “k_{d}”, to the rate of association, or “on-rate” or “kₐ”. Thus, K_{D} equals k_{d}/ kₐ and is expressed as a molar concentration (M). It follows that the smaller the K_{D}, the stronger the binding affinity. Therefore, a K_{D} of 1 μM indicates weak binding affinity compared to a K_{D} of 1 nM. K_{D} values for antibodies can be determined using methods well established in the art. One method for determining the K_{D} of an antibody is by using surface plasmon resonance, typically using a biosensor system such as a BIACORE® system.

An “antibody” or “Ab” is an immunoglobulin molecule capable of recognizing and binding to a specific target or antigen, such as a carbohydrate, polynucleotide, lipid, polypeptide, etc., through at least one antigen recognition site, located in the variable region of the immunoglobulin molecule. As used herein, the term “antibody” can encompass any type of antibody, including but not limited to monoclonal antibodies, polyclonal antibodies, “antigen-binding fragments” (or portion), such as Fab, Fab′, F(ab′)₂, Fd, Fv, Fc, etc., of intact antibodies that retain the ability to specifically bind to a given antigen (e.g. EFNA4), an isolated complementarity determining region (CDR), bispecific antibodies, heteroconjugate antibodies, mutants thereof, fusion proteins having an antibody, or antigen-binding fragment thereof, (e.g., a domain antibody), single chain (ScFv) and single domain antibodies (e.g., shark and camelid antibodies), maxibodies, minibodies, intrabodies, diabodies, triabodies, tetrabodies, v-NAR and bis-scFv (see, e.g., Holliger and Hudson, 2005, Nature Biotechnology 23(9): 1126-1136), humanized antibodies, chimeric antibodies and any other modified configuration of the immunoglobulin molecule that includes an antigen recognition site of the required specificity, including glycosylation variants of antibodies, amino acid sequence variants of antibodies, and covalently modified antibodies. The antibodies may be of murine, rat, human, or any other origin (including chimeric or humanized antibodies). In some aspects of the invention, the antibody, or antigen-binding fragment thereof, of the disclosed anti-EFNA4 antibody-drug conjugates is a chimeric, humanized, or a recombinant human antibody, or EFNA4-binding fragment thereof.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-Lazikani et al., J. Molec. Biol. 273:927-948 (1997)). As used herein, a CDR may refer to CDRs defined by either approach or by a combination of both approaches.

A CDR of a variable domain are comprised of amino acid residues within the variable region that are identified in accordance with the definitions of Kabat, Chothia, the accumulation of both Kabat and Chothia, VBASE2, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, (1989). The CDR positions may also be derived from an analysis of the VBASE2 database. (See, e.g. Retter et al., Nucleic Acids Res. 33(Database Issue):D671-D674, 2005).

Other approaches to CDR identification include the “AbM definition,” which is a compromise between Kabat and Chothia and is derived using Oxford Molecular's AbM antibody modeling software (now ACCELRYS®), or the “contact definition” of CDRs based on observed antigen contacts, set forth in MacCallum et al., J. Mol. Biol., 262:732-745, (1996). In another approach, referred to herein as the “conformational definition” of CDRs, the positions of the CDRs may be identified as the residues that make enthalpic contributions to antigen binding. See, e.g., Makabe et al., Journal of Biological Chemistry, 283:1156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For anti-EFNA4 antibody-drug conjugates described herein, CDRs may be defined in accordance with any of Kabat, Chothia, extended, VBASE2, AbM, contact, and/or conformational definitions.

In other aspects of the invention, the anti-EFNA4 antibody-drug conjugates comprise an antibody or antigen-binding fragment thereof that further comprises one or more CDR(s) of the antibody or antigen-binding fragment thereof (such as one, two, three, four, five, or all six CDRs).

Antibodies, antibody domains, and antigen-binding fragments thereof may be described as “polypeptides”, “oligopeptides”, “peptides” and “proteins”, i.e., chains of amino acids of any length, preferably, relatively short (e.g., 10-100 amino acids). The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains. Amino acids may be referred to herein by either their commonly known three letter symbols or by the one-letter symbols recommended by the IUPAC-IUB Commission on Biochemical Nomenclature.

As used herein, “humanized antibody” or “CDR grafted antibody” refers to forms of non-human (e.g. murine) antibodies that are chimeric immunoglobulins, immunoglobulin chains, or fragments thereof (such as Fv, Fab, Fab′, F(ab′)₂ or other antigen binding subsequences of antibodies) that contain minimal sequences derived from a non-human immunoglobulin. Preferably, humanized antibodies are human immunoglobulins (recipient antibody) in which residues from one or more complementarity determining regions (CDRs) of the recipient are replaced by residues from one or more CDRs of a non-human species (donor antibody) such as mouse, rat, or rabbit having the desired specificity, affinity, and capacity.

As used herein, the term “dosing regimen” refers to the total course of treatment administered to a patient, e.g., treatment with an anti-EFNA4 ADC.

As used herein, “dose limiting toxicity” (DLT) refers to the dosage of the anti-EFNA4 antibody-drug conjugate that is contraindicative of a further increase in dosage. DLT is graded according to NCI Common Terminology Criteria (v 4.03) during the first cycle of treatment which is not clearly related to disease progression. Hematologic: grade 4 neutropenia for >7 days; febrile neutropenia; grade ≥3 neutropenia with infection; thrombocytopenia with clinically significant bleeding; or grade 4 thrombocytopenia. Non-hematologic: grade ≥3 toxicities, excluding those not treated maximally; or delay by more than 2 weeks in receiving the next scheduled cycle due to persisting toxicities not attributable to disease progression.

As used herein “maximum tolerated dose” (MTD) refers to the highest dosage of the anti-EFNA4 antibody-drug conjugate that does not cause unacceptable side effects. MTD is estimated using the mTPI based on observed DLT rate, with a target DLT rate of 25% and equivalence interval of 20-30%. At least 9 patients have been accumulated at a dose that is predicted to be the MTD.

The disclosed anti-EFNA4 antibody-drug conjugates may be administered as an initial treatment, or for treatment of conditions that are unresponsive to conventional therapies. In addition, the EFNA4 antibody-drug conjugates may be used in combination with other therapies (e.g., surgical excision, radiation, additional anti-cancer drugs etc.) to thereby elicit additive or potentiated therapeutic effects and/or reduce hepatocytotoxicity of some anti-cancer agents. EFNA4 antibody-drug conjugates of the invention may be co-administered or co-formulated with additional agents, or formulated for consecutive administration with additional agents in any order.

As used herein, the phrases “effective amount” or “effective dosage” are used interchangeably and refer to an amount of a drug (e.g., anti-EFNA4 antibody-drug conjugate), compound, or pharmaceutical composition necessary to achieve one or more beneficial or desired prophylactic or therapeutic results. For prophylactic use, beneficial or desired results include eliminating or reducing the risk of developing a disease (e.g., cancer and/or EFNA4-associated disorder), delaying the onset of the disease, or preventing the progression of the disease. For therapeutic use, beneficial or desired results include eliminating, reducing the incidence of, or ameliorating one or more symptoms of, these diseases or conditions. Determination of an effective amount or dosage may include observing or measuring changes in: biochemical or histological markers; behavioral symptoms of the disease; complications of the disease; and intermediate pathological phenotypes presenting during development of the disease. Determination of an effective amount or dosage may also include observing or measuring a decrease in the dose of another drug/medication required to treat the disease; or an increase in the efficacy of another drug/medication. In particular aspects of the invention, the efficacy of treatment may be determined by measuring the decrease in tumor size as compared to the tumor size in the patient prior to the initial administration of the anti-EFNA4 antibody-drug conjugate using methods known in the art (e.g., Response Evaluation Criteria In Solid Tumors (RECIST)). For example, the tumor may decrease in size by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or up to 100% or up to a point at which the tumor is no longer detectable. In one aspect, the invention provides a method for treating a condition associated with EFNA4 expression in a patient. The invention also provides an antibody-drug conjugate, or a pharmaceutical composition, as described herein, for use in a method for treating a condition associated with EFNA4 expression in a patient. The invention further provides the use of an antibody-drug conjugate, or a pharmaceutical composition, as described herein, in the manufacture of a medicament for treating a condition associated with EFNA4 expression in a patient.

In some aspects of the invention, the method of treating a condition associated with EFNA4 expression in a patient includes administering to the patient in need thereof an effective amount of a composition (e.g., pharmaceutical composition) comprising an EFNA4 antibody-drug conjugate as described herein. The conditions associated with EFNA4 expression include, but are not limited to, abnormal EFNA4 expression, altered or aberrant EFNA4 expression, EFNA4 overexpression, and a proliferative disorder (e.g., cancer).

Cancers suitable for targeting using anti-EFNA4 antibody-drug conjugates include EFNA4-expressing primary and metastatic cancers, such as breast cancer (such as triple-negative breast cancer (TNBC)), ovarian cancer, colorectal cancer, liver cancer (such as hepatocellular carcinoma (HCC)), lung cancer (such as non-small cell lung cancer (NSCLC) and small cell lung cancer (SCLC)), appendiceal cancer, spindle cell sarcoma, pancreatic cancer, cervical adenocarcinoma, peritoneal cancer, metastatic melanoma, renal cancer, neoplasm peritoneum, and leukemias such as chronic lymphocytic leukemia-CLL.

In some aspects of the invention, provided is a method of inhibiting tumor growth or progression in a patient who has a EFNA4 expressing tumor, including administering to the patient in need thereof an effective amount of a composition having the EFNA4 antibody-drug conjugates as described herein. In other aspects of the invention, provided is a method of inhibiting metastasis of EFNA4 expressing cancer cells in a patient, including administering to the patient in need thereof an effective amount of a composition having the EFNA4 antibody-drug conjugates as described herein. In other aspects of the invention, provided is a method of inducing regression of a EFNA4 expressing tumor regression in a patient, including administering to the patient in need thereof an effective amount of a composition having the EFNA4 antibody-drug conjugates as described herein. In other aspects, the invention provides an antibody-drug conjugate, or a pharmaceutical composition, as described herein, for use in a method as described above. In other aspects, the invention provides the use of an antibody-drug conjugate, or a pharmaceutical composition, as described herein, in the manufacture of a medicament for use in the methods described above.

As used herein, the terms “individual”, “subject”, and “patient” are used interchangeably and refer to a mammal, including, but not limited to, farm animals, sport animals, pets, primates, horses, dogs, cats, mice and rats. In a preferred aspect of the invention, the mammal is a human.

As used herein, the terms “pharmaceutically acceptable carrier” and “pharmaceutical acceptable excipient” are used interchangeably and refer to any material which, when combined with an active ingredient, allows the ingredient to retain biological activity and is non-reactive with the patient's immune system. Examples include, but are not limited to, any of the standard pharmaceutical carriers such as a phosphate buffered saline solution, water, emulsions such as oil/water emulsion, and various types of wetting agents. Compositions comprising such carriers are formulated by well-known conventional methods (see, for example, Remington's Pharmaceutical Sciences, 18^{th} edition, A. Gennaro, ed., Mack Publishing Co., Easton, Pa., 1990; and Remington, The Science and Practice of Pharmacy, 20th Ed., Mack Publishing, 2000).

Reference to “about” a value or parameter herein includes (and describes) embodiments that are directed to that value or parameter per se. For example, description referring to “about X” includes description of “X.” Numeric ranges are inclusive of the numbers defining the range.

It is understood that wherever embodiments are described herein with the language “comprising,” otherwise analogous embodiments described in terms of “consisting of” and/or “consisting essentially of” are also provided.

Additional scientific and technical terms used in connection with the present invention, unless indicated otherwise herein, shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, genetics, and protein and nucleic acid chemistry and hybridization described herein are those well-known and commonly used in the art.

### Dosing Regimens

The present invention provides for dosing regimens and methods for the treatment of patients with cancer and/or an EFNA4-associated disorder with an anti-EFNA4 antibody-drug conjugate (ADC). The present invention further provides for dosing regimens and methods for the treatment of patients with cancer and/or an EFNA4-associated disorder in which an anti-EFNA4 ADC is administered to a patient intravenously, subcutaneously, intramuscularly, by bolus injection, intracerebrally or by sustained release. The present invention further provides for dosing regimens and methods for the treatment of patients with cancer and/or an EFNA4-associated disorder in which an anti-EFNA4 ADC administered to a patient at least twice every week, at least weekly (QW), at least every 2 weeks (Q2W), at least every 3 weeks (Q3W) or at least every 4 weeks (Q4W). The present invention further provides for dosing regimens and methods for the treatment of patients with cancer and/or an EFNA4-associated disorder in which an anti-EFNA4 ADC is administered to a patient intravenously weekly (QW) or every 3 weeks (Q3W).

In some aspects of the invention, the dosing regimen and methods comprise administering to a patient a dose of about 0.010 mg/kg to about 10 mg/kg, about 0.010 mg/kg to about 5 mg/kg, about 0.010 mg/kg to about 1 mg/kg or about 0.010 mg/kg to about 0.500 mg/kg of the anti-EFNA4 antibody-drug conjugates. In some aspects of the invention, the anti-EFNA4 antibody-drug conjugates are administered to a patient at a dose of at least 0.010, 0.015, 0.020, 0.025, 0.030, 0.035, 0.040, 0.045, 0.050, 0.055, 0.060, 0.065, 0.070, 0.075, 0.080, 0.095, 0.100, 0.150, 0.200, 0.250, 0.300, 0.350, 0.400, 0.450 or 0.500 mg/kg. In some aspects of the invention, the anti-EFNA4 antibody-drug conjugates are administered to a patient at a dose of about 0.010 mg/kg, 0.015 mg/kg, 0.020 mg/kg, 0.030 mg/kg, 0.050 mg/kg, 0.075 mg/kg, 0.100 mg/kg, or 0.134 mg/kg. In another aspects of the invention, the anti-EFNA4 antibody-drug conjugate are administered weekly (QW) at a dose of about 0.01 mg/kg, 0.015 mg/kg or 0.02 mg/kg. In another aspect of the invention, the anti-EFNA4 antibody-drug conjugates are administered to a patient every 3 weeks (Q3W) at a dose of about 0.015 mg/kg, 0.030 mg/kg, 0.050 mg/kg, 0.075 mg/kg, 0.100 mg/kg, or 0.134 mg/kg.

The present invention further provides for dosing regimens and methods for the treatment of patients with cancer and/or an EFNA4-associated disorder in which the treatment results in a decrease in a tumor size of at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% as compared to the tumor size in the patient prior to initial administration of the anti-EFNA4 antibody-drug conjugate. A decrease in tumor size may be measured or determined by any method used and accepted in the art (e.g., RECIST v.1.1).

### Anti-EFNA4 Antibody-Drug Conjugates (ADCs)

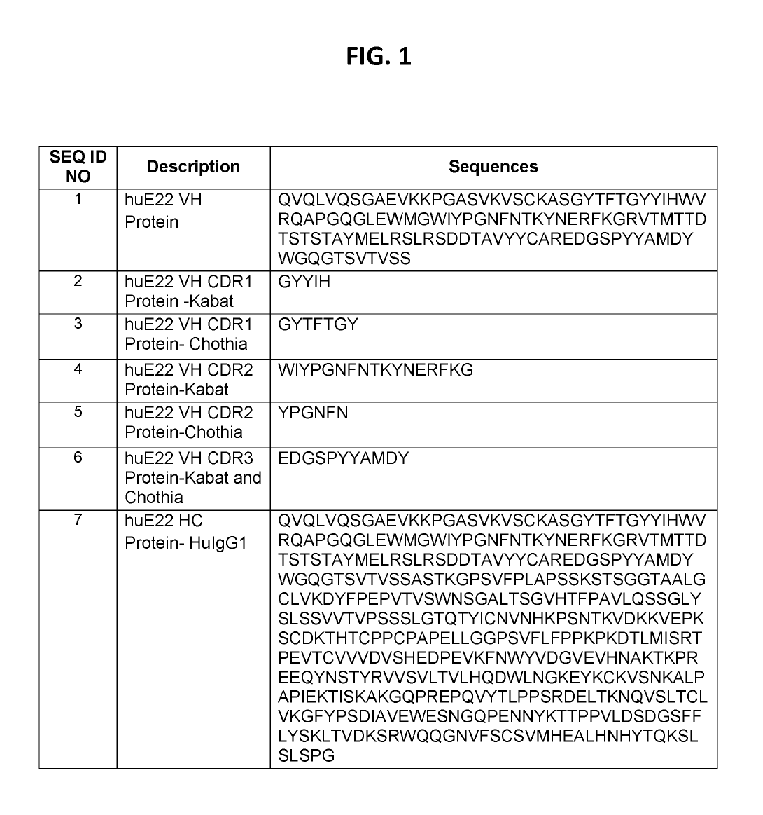

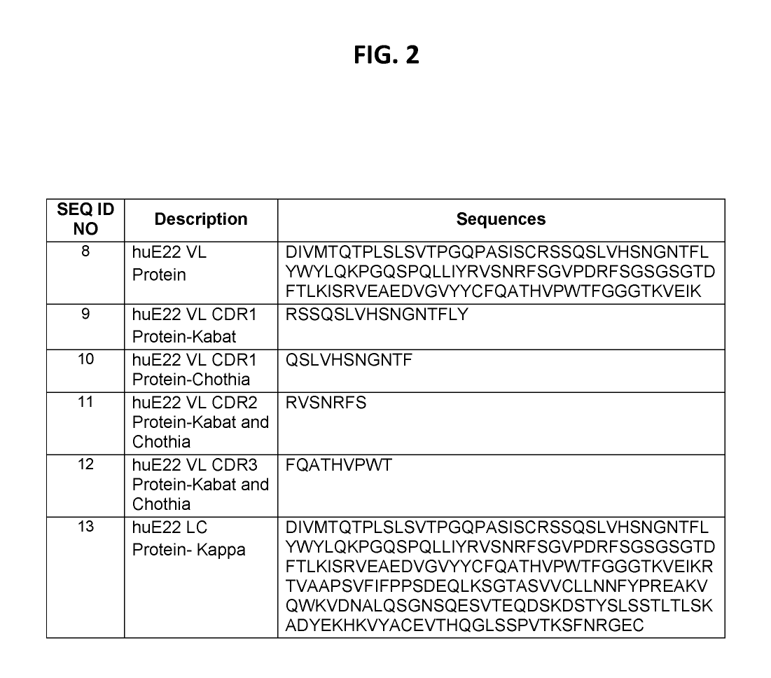

The invention can be practiced using, for example, an anti-EFNA4 ADC comprising an antibody that specifically binds to human EFNA4. In some aspects of the invention, the invention can be practiced with an anti-EFNA4 ADC comprising an antibody comprising three CDRS from a heavy chain variable region (VH) having the amino acid sequence shown in SEQ ID NO: 1 and three CDRS from a light chain variable region (VL) having the amino acid sequence shown in SEQ ID NO: 8. In another aspect of the invention, the anti-EFNA4 ADC comprises an antibody comprising a VH CDR1 having the amino acid sequence shown in SEQ ID NO: 2, VH CDR2 having the amino acid sequence shown in SEQ ID NO: 4, and VH CDR3 having the amino acid sequence shown in SEQ ID NO: 6, and/or VL CDR1 having the amino acid sequence shown in SEQ ID NO: 9, VL CDR2 having the amino acid sequence shown in SEQ ID NO: 11, and VL CDR3 having the amino acid sequence shown in SEQ ID NO: 12. In some aspects of the invention, the anti-EFNA4 antibody-drug conjugates comprise an antibody comprising a heavy chain variable region (VH) having the amino acid sequence shown in SEQ ID NO: 1 and a light chain variable region (VL) having the amino acid sequence shown in SEQ ID NO: 8. In some aspects of the invention, the anti-EFNA4 ADC comprises an antibody designated huE22. See FIGS. 1 and 2.

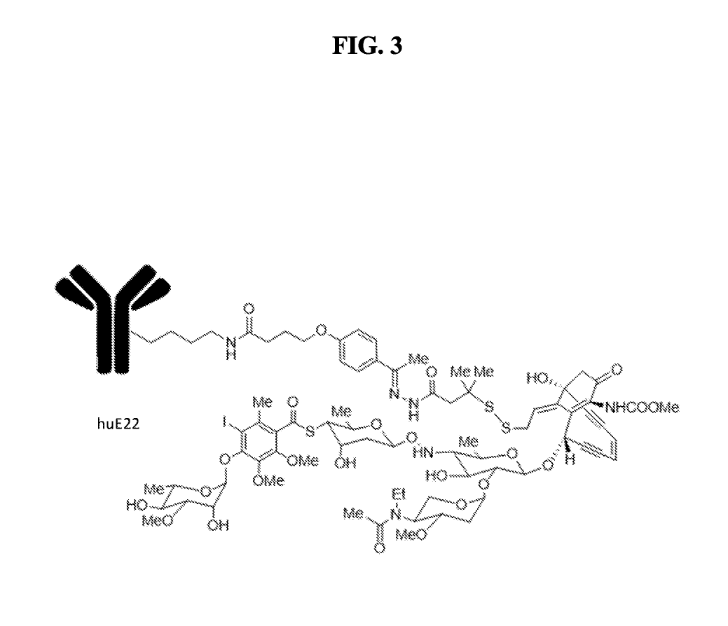

In some aspects, the invention can be practiced with an anti-EFNA4 ADC having a cytotoxic drug conjugated to an anti-EFNA4 antibody. In one aspect, the invention can be practiced with an anti-EFNA4 ADC having the drug calicheamicin or a calicheamicin derivative (CM) conjugated to the anti-EFNA4 antibody. In a further aspect, the invention can be practiced with an anti-EFNA4 ADC designated huE22-AcBut-CM, which comprises the antibody designated huE22 conjugated to CM via a 4-(4′acetylphenoxy)butanoic acid (AcBut) linker, termed huE22-AcBut-CM. See FIGS. 3A and 3B.

These anti-EFNA4 antibodies and ADCs are described in U.S. Patent Publication No. 2015/0125472 and International Patent Application Publication WO 2012/118547, each of which is herein incorporated by reference in its entirety.

### EFNA4-Associated Disorders

In other aspects, the cancer and/or EFNA4-associated disorder includes hyperproliferative disorders, such as neoplastic disorders, such as solid tumors, e.g., breast cancer (such as triple-negative breast cancer (TNBC)), ovarian cancer, colorectal cancer, liver cancer (such as hepatocellular carcinoma (HCC)), lung cancer (such as non-small cell lung cancer (NSCLC) and small cell lung cancer (SCLC)), appendiceal cancer, spindle cell sarcoma, pancreatic cancer, cervical adenocarcinoma, peritoneal cancer, metastatic melanoma, renal cancer, neoplasm peritoneum, etc., and hematologic malignancies, e.g., leukemias (such as chronic lymphocytic leukemia (CLL)), etc. In another aspect, the cancer and/or EFNA4-associated disorder is characterized by the overexpression of EFNA4.

### Pharmaceutical Compositions

Further provided herein are pharmaceutical compositions comprising anti-EFNA4 ADCs disclosed herein and a pharmaceutically acceptable carrier. The present invention also provides articles of manufacture, comprising a container, a composition within the container comprising an anti-EFNA4 ADC, and a package insert containing instructions to administer a dose of anti-EFNA4 ADC.

Another aspect of the invention provides for kits containing a formulation comprising a pharmaceutical composition. The kits may comprise an anti-EFNA4 ADC and a pharmaceutically acceptable carrier. The kits may contain instructions for QW and/or Q3W intravenous dosing of the pharmaceutical composition for the treatment of cancer and/or an EFNA4-associated disorder in which the administration of an anti-EFNA4 ADC is beneficial.

### EXAMPLES

The following examples are meant to illustrate the methods and materials of the present invention. Suitable modifications and adaptations of the described conditions and parameters normally encountered in the art that are obvious to those skilled in the art are within the spirit and scope of the present invention.

### Example 1

### Anti-EFNA4 huE22-AcBut-CM ADC Study Overview

### A. Objectives

This example illustrates a Phase 1, two part, open-label, unblinded, multi-center, single arm, non-randomized, multiple dose, safety, pharmacokinetic (PK) and pharmacodynamic (PD) study of single agent huE22-AcBut-CM ADC in sequential cohorts of adult patients with advanced solid tumors for whom no standard therapy is available.

The primary objective of Part 1 was to assess safety and tolerability of huE22-AcBut-CM ADC in order to estimate the maximum tolerated dose (MTD) and select the recommended Phase 2 dose (RP2D). The secondary objectives included: evaluate the overall safety profile, characterize single and multiple dose pharmacokinetics, total antibody and unconjugated payload, evaluate the immunogenicity of huE22-AcBut-CM ADC and document preliminary evidence of efficacy and anti-tumor activity based on the response rate (RR).

The primary objective of Part 2 is to confirm the safety and tolerability and evaluate evidence of efficacy and anti-tumor activity of huE22-AcBut-CM ADC based on RR.

### B. Treatment Schedule

In the dose-escalation Part 1, huE22-AcBut-CM ADC was administered intravenously (IV) as a single agent to adult patients with advanced solid tumors. Dose escalation was studied in cohorts of 2-4 patients administered huE22-AcBut-CM ADC by IV every 3 weeks (Q3W) or weekly (QW). A modified toxicity probability interval method (mTPI) targeting a dose-limiting toxicity (DLT) rate of 25% with an equivalence interval (20%-30%) was utilized in order to estimate MTD. The starting dose level for each regimen was 0.015 mg/kg Q3W and 0.01 mg/kg QW. Subsequent dose levels may include a maximum 100% escalation until either the dose is ≥0.060 mg/kg, a patient experiences a DLT or Grade 2 thrombocytopenia considered related to huE22-AcBut-CM ADC after which, dose escalation in subsequent cohorts would follow a modified Fibonacci scheme.

Evaluation of the QW regimen was scheduled to be initiated once a patient treated in the Q3W regimen experienced a DLT or Grade 2 thrombocytopenia related to the study treatment related to the study treatment at a starting dose of ≤33% of the highest cumulative dose that has been evaluated in the Q3W regimen. In the Q3W regimen, 0.015, 0.03, 0.05, 0.075, 0.1 and 0.134 mg/kg were evaluated. A patient experienced Grade 2 thrombocytopenia considered related to huE22-AcBut-CM ADC, thus the QW regimen was initiated at 0.01 mg/kg. In the QW regimen, 0.01, 0.015, and 0.02 mg/kg were evaluated.

Patients participated in the study for approximately 6 months. This 6-month period included up to 4 weeks of screening, approximately 4 months of treatment, and a follow-up visit within 4 weeks after the last dose for adverse event (AE) and serious adverse event (SAE) collection. huE22-AcBut-CM ADC was administered per the Dosage Administration Instruction (DAI) as an IV infusion over approximately 60 minutes (±5 minutes) on an outpatient basis. Treatment with study drug continued until disease progression, patient refusal, unacceptable toxicity occurred, or the study was terminated.

In the dose-expansion Part 2, huE22-AcBut-CM ADC is administered intravenously (IV) as a single agent to adult patients with triple-negative breast cancer (TNBC) or EFNA4-expressing ovarian cancer. The RP2D was identified from Part 1 data provided herein to be 0.015 mg/kg weekly (QW).

### C. Patient Population

All patients being considered for the study and eligible for screening were required to sign an informed consent for the study before completing any study-specific procedures. Key inclusion criteria for Part 1 included: adult patient (age ≥18 years) with histological or cytological diagnosis of solid tumor that was advanced/metastatic and resistant to standard therapy or for which no standard therapy was available, and adequate bone marrow, renal, and liver function. Patients were excluded from this study if they met the following key exclusion criteria: no primary or metastatic central nervous system tumor involvement, and major surgery or systemic anticancer therapy within 4 weeks of starting treatment.

Key inclusion criteria for Part 2 includes: adult patients (age ≥18 years) with previously treated EFNA4 expressing triple-negative breast cancer (TNBC) or ovarian cancer (OVCA), and measurable disease per Response Evaluation Criteria In Solid Tumors (RECIST) v1.1.

### D. Assessments

Safety assessments included collection of AEs, SAEs, vital signs and physical examination, ECG (12 lead), laboratory assessments, including pregnancy tests and verification of concurrent medications.

Pharmacokinetic assessments included serum concentrations of huE22-AcBut-CM ADC (measured as conjugated payload), total antibody, and unconjugated payload were quantified using validated bioanalytical assays. Specifically, total antibody concentrations were measured using ELISA method, huE22-AcBut-CM ADC concentrations were measured as conjugated payload using a hybrid LC-MS/MS method, and unconjugated payload concentrations will be measured using an LC-MS/MS method. For preliminary PK assessment, mean serum concentration-time profiles of huE22-AcBut-CM ADC were generated for each dose cohort; Noncompartmental PK parameters were estimated from Cycle 1 concentration-time data using nominal sampling time. For huE22-AcBut-CM ADC and total antibody, PK parameters including the maximum plasma concentration (Cmax), time to maximum plasma concentration (Tmax), and area under the plasma concentration versus time curve (AUC_{inf}, AUCT), clearance (CL), volume of distribution at steady state (Vss), terminal half-life (t_{1/2}), and accumulation ratio (R_{ac}) were calculated. For unconjugated payload, PK parameters including Cmax, Tmax, AUCi_{nf}, AUCT, t_{1/2}, and R_{ac} were calculated.

Response: Tumors were assessed at baseline and every 6 weeks while on-study and responses were characterized according to RECIST 1.1. Changes in tumor size was categorized as complete response (CR), partial response (PR), stable disease (SD), or progressive disease (PD), the latter incorporating the appearance of new lesions.

### Example 2

### Anti-EFNA4 huE22-AcBut-CM ADC Part 1 Study Results

Data were obtained for a total of 48 patients with solid tumors treated with huE22-AcBut-CM ADC either once every 3 weeks (25 Q3W patients) or once weekly (23 QW patients) in the Phase 1, Part 1 dose escalation study. The demographic and baseline characteristics of the patients included in this study are listed in Table 1.

TABLE 1
Demographics and baseline characteristics.

| | Q3W | | QW | |
|---|---|---|---|---|
| | N = 25 | | N = 23 | |
| Age, Mean years (range) | 58.6 | (35-82) | 61.3 | (40-77) |

Gender

| | | | | |
|---|---|---|---|---|
| Female | 20 | (80%) | 22 | (96%) |
| Male | 5 | (20%) | 1 | (4%) |

Race

| | | | | |
|---|---|---|---|---|
| White | 18 | (72%) | 19 | (83%) |
| Black | 2 | (8%) | 4 | (17%) |

Primary Tumor Type

| Triple negative breast cancer (TNBC) | 4 | (16%) | 4 | (17%) |
|---|---|---|---|---|
| Ovarian cancer (OVCA) | 5 | (20%) | 11 | (48%) |
| Breast (Not TNBC) | 3 | (12%) | 2 | (9%) |
| Peritoneal cancer | 2 | (8%) | 0 | |
| Other*/Other** | 11 | (44%) | 6 | (26%) |

*Unknown primary carcinoma, cervical cancer, colon cancer, endometrial cancer, melanoma, rectal cancer, renal cancer, squamous cell carcinoma of the lung, medullary carcinoma
**Appendiceal cancer, urothelial carcinoma of the bladder, carcinoma of the lung, pancreatic cancer, sarcoma of the right buttock, uterine cancer

Table 2 provides the number of patients dosed and the DLTs observed for each Q3W and QW regimen. The doses ranged from 0.015 to 0.134 mg/kg Q3W and 0.01 to 0.02 mg/kg QW. huE22-AcBut-CM ADC administered Q3W and QW had a manageable toxicity profile in patients with advanced solid malignancies.

TABLE 2
Dose escalation.

| | huE22-AcBut- | | |
|---|---|---|---|
| | CM ADC | Patients | |
| Regimen | Dose (mg/kg) | treated | DLT |
| Q3W | 0.015 | 2 | 0 |
| N = 25 | 0.03 | 3 | 0 |
| | 0.05 | 9 | 2 (Thrombocytopenia) |
| | 0.075 | 3 | 0 |
| | 0.100 | 6 | 2 (Thrombocytopenia) |
| | 0.134 | 2 | 2 (Aspartate transferase elevation/ |
| | | | epistaxis, Stomatitis) |
| QW | 0.01 | 3 | 0 |
| N = 23 | 0.015 | 13 | 1 (Death*) |
| | 0.02 | 7 | 1 (Thrombocytopenia) |

*Death (cause undetermined) 1 week after 1^{st} dose

Table 3 provides the current patient disposition of the 48 study patients.

TABLE 3
Patient disposition.

| | Q3W | QW |
|---|---|---|
| | N = 25 | N = 23 |
| Continuing on-study treatment | 0 | 1 |
| Discontinued study treatment | 25 | 22 |
| Reasons for discontinuation | | |
| Objective disease progression or relapse | 10 | 11 |
| Adverse event | 5 | 3 |
| Withdrawal of patient consent | 5 | 3 |
| Global deterioration of health status | 3 | 3 |
| Death | 1 | 1 |
| Other | 1* | 1** |

*decreased quality of life;
**patient referred for mastectomy

Table 4 provides the huE22-AcBut-CM ADC exposure of the 48 patients. The total number of administered cycles was 82 (Q3W) and 81 (QW) during the first cycle. The * indicates the patient is still on treatment.

TABLE 4
huE22-AcBut-CM ADC exposure.

| Q3W Regimen | | | QW Regimen | | |
|---|---|---|---|---|---|
| | Median Days | | | Median Days | |
| Dose Level | of Treatment | Number of | Dose Level | of Treatment | Number of |
| (mg/kg) | (range) | Cycles | (mg/kg) | (range) | Cycles |
| 0.015 (n = 2) | 23 (22-23) | 2, 2 | 0.01 (n = 3) | 78 (43-136) | 2, 4, 7 |
| 0.03 (n = 3) | 85 (23-147) | 2, 5, 8 | 0.015 (n = 13) | 15 (1-30) | 1, 1, 2, 2, 2, |
| | | | | | 3, 4, 4, 4, 5*, |
| | | | | | 5, 6, 9 |
| 0.05 (n = 9) | 120 (1-211) | 1, 1, 1, 1, 2, | 0.02 (n = 7) | 28 (8-155) | 1, 1, 1, 2, 2, 2, |
| | | 2, 6, 7, 9 | | | 11 |
| 0.075 (n = 3) | 64 (24-148) | 2, 4, 7 | Total | | |
| | | | (n = 23) | | |
| 0.10 (n = 6) | 45 (1-134) | 1, 1, 2, 2, 4, 6 | | | |
| 0.134 (n = 2) | 23 (22-24) | 2, 2 | | | |
| Total | | | | | |
| (n = 25) | | | | | |

Tables 5 summarizes the treatment-emergent adverse events (AEs) occurring in ≥20% of patients at all dose levels for the QW regimen of huE22-AcBut-CM ADC. Table 6 summarizes the treatment-emergent AEs occurring in ≥20% of patients at all dose levels for the Q3W regimen of huE22-AcBut-CM ADC. Common treated-emergent AEs were fatigue, nausea, vomiting, decreased appetite, diarrhea, dysgeusia mucosal inflammation, constipation and thrombocytopenia (TCP). For QW, the number of patients that experienced AEs with maximum severity of Grade 2 were: 11/23 (48%) for all causality and 10/21 (44%) for treatment related. For Q3W, the number of patients that experienced AEs with maximum severity of Grade 2 were: 7/25 (28%) for all causality and 11/24 (44%) for treatment related.

TABLE 5
Treatment-emergent AEs (≥20%) QW (n = 23).

| | All Causality (%) | | Treatment-Related (%) | |
|---|---|---|---|---|
| | All Grades | Grade 3* | All Grades | Grade 3** |
| Fatigue | 18 (78) | 2 (9) | 16 (70) | 2 (9) |
| Nausea | 15 (65) | 0 | 13 (57) | 0 |
| Vomiting | 13 (57) | 0 | 10 (44) | 0 |
| Decreased | 10 (44) | 0 | 7 (30) | 0 |
| appetite | | | | |
| Diarrhea | 10 (44) | 1 (4) | 7 (30) | 0 |
| Dysgeusia | 9 (39) | 0 | 7 (30) | 0 |
| Mucosal | 9 (39) | 1 | 8 (35) | 1 (4.3) |
| inflammation | | | | |
| Constipation | 7 (30) | 0 | 0 | 0 |
| Thrombocytopenia | 7 (30) | 1 (4) | 7 (30) | 1 (4.3) |
| Abdominal pain | 6 (26) | 1 (4) | 0 | 0 |
| Dyspnea | 6 (26) | 0 | 0 | 0 |
| Alopecia | 5 (22) | 0 | 5 (22) | 0 |
| Pyrexia | 5 (22) | 1 (4) | 0 | 0 |

*Grade 4: Gastritis (n = 1); Grade 5: Death (n = 1), Disease progression (n = 1)
**Grade 4: Gastritis (n = 1); Grade 5: Death (n = 1)

TABLE 6
Treatment-emergent AEs (≥20%) Q3W (n = 25).

| | All Causality (%) | | Treatment-Related (%) | |
|---|---|---|---|---|
| | All Grades | Grade 3* | All Grades | Grade 3** |
| Fatigue | 17 (68) | 3 (12) | 15 (60) | 1 (4) |
| Nausea | 17 (68) | 1 (4) | 14 (56) | 0 |
| Decreased | 15 (60) | 0 | 11(44) | 0 |
| appetite | | | | |
| Thrombocytopenia | 11 (44) | 1 (4) | 11(44) | 1 (4) |
| Dysgeusia | 9 (36) | 0 | 9 (36) | 0 |
| Abdominal | 8 (32) | 0 | 0 | 0 |
| Diarrhea | 8 (32) | 1 (4) | 7 (28) | 0 |
| Mucosal | 8 (32) | 3 (12) | 8 (32) | 3 (12) |
| inflammation | | | | |
| Vomiting | 8 (32) | 1 (4) | 7 (28) | 0 |
| Skin | 7 (28) | 0 | 7 (28) | 0 |
| hyperpigmentation | | | | |
| Stomatitis | 7 (28) | 1 (4) | 7 (28) | 1 (4) |
| Constipation | 6 (24) | 0 | 0 | 0 |
| Dry mouth | 6 (24) | 0 | 5 (20) | 0 |
| Dyspnea | 6 (24) | 1 (4) | 0 | 0 |
| Edema peripheral | 6 (24) | 0 | 0 | 0 |
| Back pain | 5 (20) | 0 | 0 | 0 |
| Headache | 5 (20) | 0 | 0 | 0 |
| Lacrimation | 5 (20) | 0 | 0 | 0 |
| increased | | | | |
| Oropharyngeal | 5 (20) | 0 | 0 | 0 |
| pain | | | | |
| Pyrexia | 5 (20) | 0 | 0 | 0 |
| Rash | 5 (20) | 0 | 5 (20) | 0 |
| Weight decreased | 5 (20) | 0 | 0 | 0 |

*Grade 4: Thrombocytopenia (n = 3), Neutropenia (n = 1), Dyspnea (n = 1); Grade 5: Disease progression (n = 2)
**Grade 4: Thrombocytopenia (n = 3) Neutropenia (n = 1)

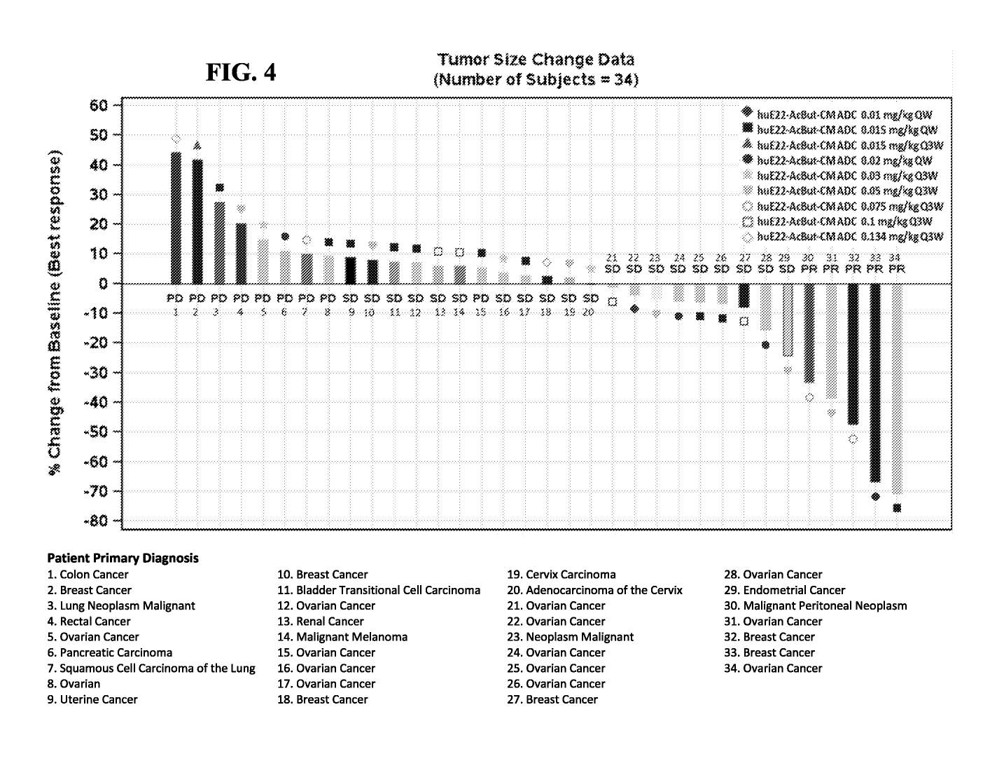

Table 7 and Table 8 provide an efficacy summary for huE22-AcBut-CM ADC treatment demonstrating the best overall response (RECIST 1.1). The data demonstrates efficacy including PRs and extended duration on-study in heavily pre-treated patients with unknown EFNA4 tumor expression in indications which are planned for expansion in Part 2 of the ongoing trial. FIG. 4 shows the maximum percentage change in tumor size for target lesions by RECIST in patients treated with huE22-AcBut-CM ADC

TABLE 7
Efficacy summary and best overall response.

| | QW Regimen | Q3W Regimen | Total |
|---|---|---|---|
| | N = 23 | N = 25 | N = 48 |
| | n (%) | n (%) | n (%) |
| Complete response | 0 | 0 | 0 |
| Partial response | 2 (9%)* | 3 (12%)** | 5 (10%) |
| Stable/No response | 12 (52%) | 11 (44%) | 23 (48%) |
| Objective progression | 6 (26%) | 5 (20%) | 11 (23%) |
| Symptomatic | 0 | 1 (4%) | 1 (2%) |
| deterioration | | | |
| Early death | 1 (4%) | 1 (4%) | 2 (4%) |
| Indeterminate | 2 (9%) | 4 (16%) | 6 (12.5%) |
| Objective Response Rate | 2 (9%) | 3 (12%) | 5 (10.4%) |
| (CR + PR) | | | |

*TNBC, OVCA
**TNBC, Peritoneal, OVCA

TABLE 8
Extended on-study duration (≥90 days).

| | | | Duration |
|---|---|---|---|
| | Dose Level | | of Therapy |
| Regimen | (mg/kg) | Primary Diagnosis | (days) |
| Q3WK | 0.03 | Adenocarcinoma Of The Cervix | 147 |
| | 0.05 | Ovarian Cancer (OVCA) | 211 |
| | 0.05 | Cervix Carcinoma | 120 |
| | 0.05 | Endometrial Cancer | 134 |
| | 0.075 | Peritoneal Cancer | 148 |
| | 0.1 | Ovarian Cancer (OVCA) | 134 |
| QW | 0.01 | Ovarian Cancer (OVCA) | 136 |
| | 0.015 | Bladder Transitional Ca | 106 |
| | 0.015 | Ovarian Cancer (OVCA) | 93* |
| | 0.015 | Ovarian Cancer (OVCA) | 99 |
| | 0.015 | Ovarian Cancer (OVCA) | 98 |
| | 0.015 | Ovarian Cancer (OVCA) | 192 |
| | 0.02 | Triple negative breast cancer | 253 |
| | | (TNBC) | |

*indicates the patient is still on treatment

### Example 3

### Anti-EFNA4 ADC Cycle-1 Serum Pharmacokinetics

Preliminary pharmacokinetic (PK) data were available for patients enrolled in six cohorts in the Q3W (dosed every 21 days) regimen (0.015, 0.03, 0.05, 0.075, 0.1 and 0.134 mg/kg) and three cohorts in the QW (dosed every 7 days) regimen (0.01, 0.015 and 0.02 mg/kg).

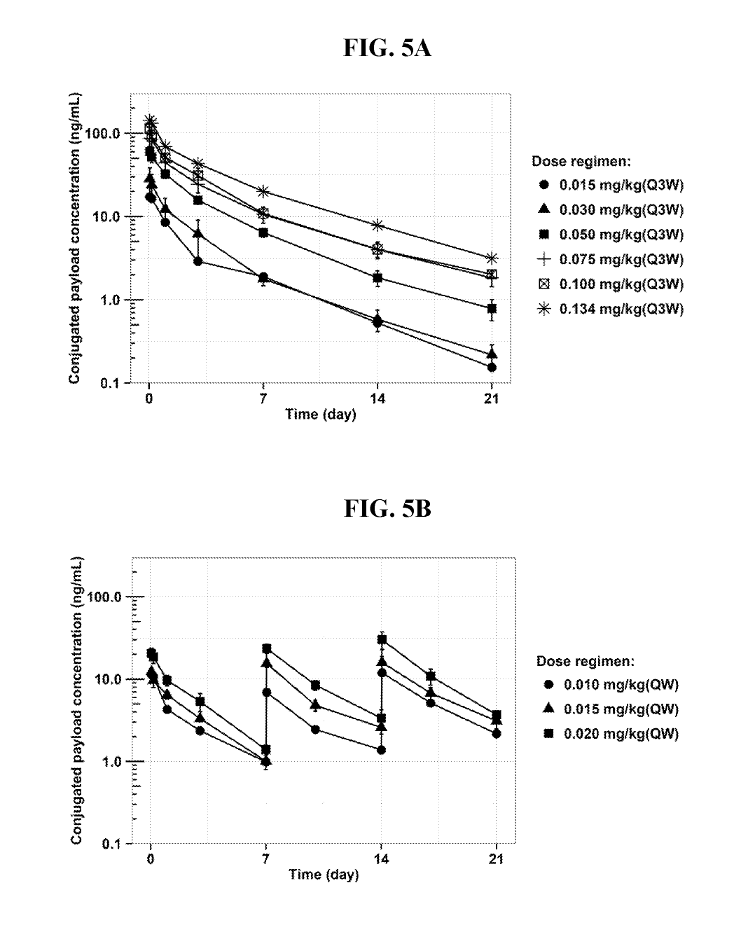

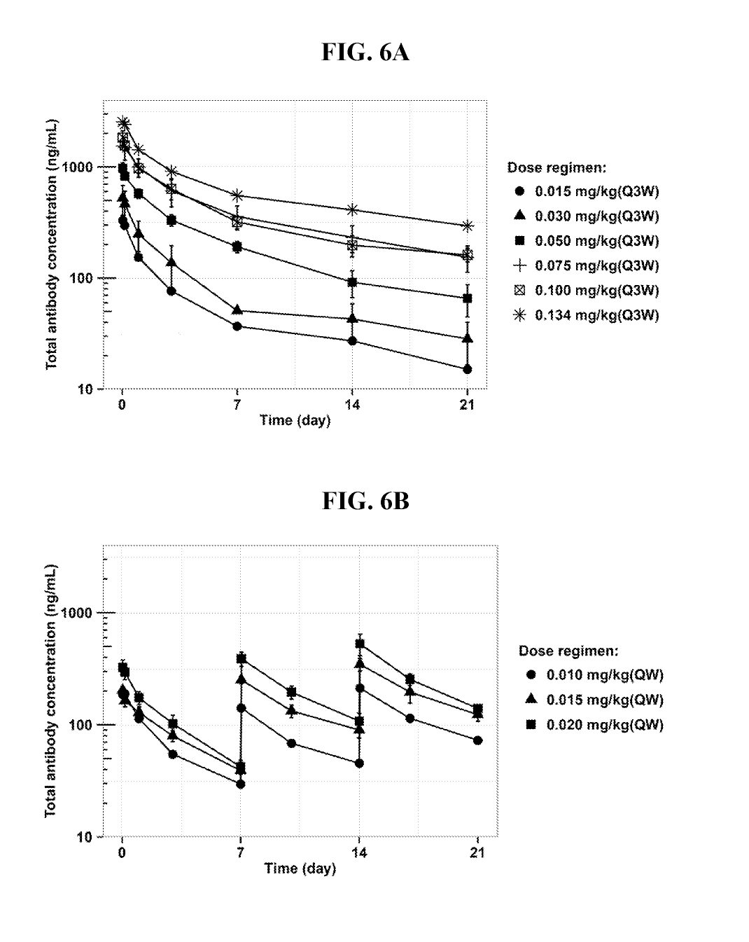

Three PK analytes were measured for PK characterization: huE22-AcBut-CM ADC (measured and reported as the concentration of antibody-conjugated payload), total antibody, and unconjugated payload. Preliminary cycle 1 antibody-conjugated payload serum concentration-time profiles for Q3W and QW dose levels are shown in FIG. 5A and FIG. 5B, respectively. Preliminary cycle 1 total antibody serum concentration-time profiles for Q3W and QW dose levels are shown in FIG. 6A and FIG. 6B, respectively. Table 9 and Table 10 contain preliminary cycle 1 PK parameters for conjugated payload and total antibody, respectively, for all dose levels. Data are presented as mean (% coefficient of variation), with the exception of Tmax, which is presented as median (range). Cycle 1 is defined as a 21-day period after first dose for both Q3W and QW regimen. Three doses are administered during cycle 1 for QW regimen and 1 dose for Q3W regimen. PK parameters were estimated using non-compartmental analysis based on the nominal sample collection time.

Abbreviations include: AUCt=area under the concentration-time profile over the first cycle (t=21 days) up to the last observable point; Cmax=maximum observed concentration over the first cycle; n=number of patients with 1 hr or 4 hr time point data after each dose in cycle 1; t½=terminal phase half-life; Tmax=time post dose at which Cmax occurs, range of Tmax values are also provided for each cohort in parenthesis; and ND=not determined.

TABLE 9
Summary of cycle 1 antibody-conjugated payload PK parameters.

| | huE22-AcBut- | | | | | |
|---|---|---|---|---|---|---|
| | CM ADC | Patients | Cmax | Tmax | AUCt | t½ |
| Regimen | Dose (mg/kg) | treated | (ug/mL) | (h) | (day · ng/mL) | (day) |
| Q3W | 0.015 | 2 | 17.3 | 2.5 | 41.3 | 5.34^{a} |
| | | | | (1.0-4.0) | | |
| | 0.03 | 3 | 28.5 | 1.0 | 64.1 | 4.54 |
| | | | (59.7%) | (1.0-1.0) | (61.2%) | (12.5%) |
| | 0.05 | 9 | 62.9 | 1.0 | 164 | 3.62 |
| | | | (40.3%) | (1.0-4.0) | (36.7%) | (25.7%) |
| | 0.075 | 3 | 89.0 | 4.0 | 276 | 5.21 |
| | | | (38.3%) | (1.0-4.0) | (36.2%) | (13.1%) |
| | 0.1 | 5 | 112 | 1.0 | 372 | 4.63 |
| | | | (32.7%) | (1.0-1.0) | (57.0%) | (26.6%) |
| | 0.134 | 2 | 143 | 1.0 | 478 | 5.48 |
| | | | | (1.0-1.0) | | |
| QW | 0.01 | 2 | 13.5 | 1.0 | 82.9 | ND |
| | | | | (1.0-1.0) | | |
| | 0.015 | 8 | 17.7 | 1.0 | 128 | ND |
| | | | (34.5%) | (1.0-4.0) | (28.6%) | |
| | 0.02 | 4 | 31.8 | 1.0 | 200 | ND |
| | | | (41.0%) | (1.0-1.0) | (23.6%) | |

^{a}Value based on n = 1 patient

TABLE 10
Summary of cycle 1 total antibody PK parameters.

| | huE22-AcBut- | | | | | |
|---|---|---|---|---|---|---|
| | CM ADC | Patients | Cmax | Tmax | AUCt | t½ |
| Regimen | Dose (mg/kg) | treated | (ug/mL) | (h) | (day · ng/mL) | (day) |
| Q3W | 0.015 | 2 | 332 | 1.0 | 1050 | 13.3^{a} |
| | | | | (1.0-1.0) | | |
| | 0.03 | 3 | 525 | 1.0 | 1960 | 8.12 |
| | | | (51.9%) | (1.0-1.0) | (67.4%) | (14.1%) |
| | 0.05 | 9 | 1000 | 1.0 | 3600 | 6.57 |
| | | | (32.1%) | (1.0-4.0) | (39.0%) | (44.0%)^{b} |
| | 0.075 | 3 | 1530 | 1.0 | 8180 | 11.2 |
| | | | (41.9%) | (1.0-4.0) | (42.7%) | (25.9%) |
| | 0.1 | 5 | 1860 | 1.0 | 8780 | 9.14 |
| | | | (32.0%) | (1.0-4.0) | (46.7%) | (35.1%) |
| | 0.134 | 2 | 2570 | 2.5 | 13100 | 15.0 |
| | | | | (1.0-4.0) | | |
| QW | 0.01 | 2 | 219 | 1.0 | 1900 | ND |
| | | | | (1.0-1.0) | | |
| | 0.015 | 8 | 352 | 1.0 | 3120 | ND |
| | | | (32.1%) | (1.0-1.0) | (28.0%) | |
| | 0.02 | 4 | 552 | 1.0 | 4040 | ND |
| | | | (37.6%) | (1.0-1.0) | (21.8%) | |

^{a}Value based on n = 1 patient;
^{b}Value based on n = 7 patients

Following single IV infusion of huE22-AcBut-CM ADC, antibody-conjugated payload and total antibody peak concentrations occurred at or shortly after the end of the infusion, followed by a decline in concentrations exhibiting multiphasic disposition characteristics for both analytes (FIGS. 5A, 5B, 6A and 6B). Antibody-conjugated payload and total antibody Cmax and exposure (AUCt) during the first cycle following huE22-AcBut-CM ADC administration increased with increasing dose (Table 9 and Table 10) in both Q3W and QW cohorts. For similar total dose over a 21 day cycle, fractionated dosing (QW) resulted in reduced Cmax levels and comparable cycle 1 exposures compared to unfractionated dosing (Table 9 and Table 10). Preliminary mean half-life (t½) for antibody-conjugated payload was estimated to be between 3.62 and 5.48 days across the dose levels tested.

Systemic exposure of unconjugated payload were generally low with the large majority of the samples tested across all dose levels observed to be below the lower limit of quantification (LLOQ, 50 pg/mL). Nearly all of the samples tested at RP2D were below the LLOQ; peak serum concentrations of unconjugated payload were <300 pg/mL across all dose levels.

What is claimed is:

1. An anti-EFNA4 antibody-drug conjugate for use in the treatment of cancer and/or EFNA4-associated disorders in a patient, wherein the anti-EFNA4 antibody-drug conjugate is administrable at least twice every week, at least weekly (QW), at least every 2 weeks (Q2W), at least every 3 weeks (Q3W) or at least every 4 weeks (Q4W).

2. The anti-EFNA4 antibody-drug conjugate for use of claim 1, wherein the anti-EFNA4 antibody-drug conjugate is administrable weekly (QW) or every 3 weeks (Q3W).

3. The anti-EFNA4 antibody-drug conjugate for use of claim 1 or 2, wherein the anti-EFNA4 antibody-drug conjugate is administrable at a dose of about 0.010 mg/kg to about 10 mg/kg, about 0.010 mg/kg to about 5 mg/kg, about 0.010 mg/kg to about 1 mg/kg or about 0.010 mg/kg to about 0.500 mg/kg.

4. The anti-EFNA4 antibody-drug conjugate for use of any one of claims 1 to 3, wherein the anti-EFNA4 antibody-drug conjugate is administrable at a dose of at least 0.010, 0.015, 0.020, 0.025, 0.030, 0.035, 0.040, 0.045, 0.050, 0.055, 0.060, 0.065, 0.070, 0.075, 0.080, 0.095, 0.100, 0.150, 0.200, 0.250, 0.300, 0.350, 0.400, 0.450 or 0.500 mg/kg.

5. The anti-EFNA4 antibody-drug conjugate for use of any one of claims 1 to 4, wherein the anti-EFNA4 antibody-drug conjugate is administrable at a dose of about 0.010 mg/kg, 0.015 mg/kg, 0.020 mg/kg, 0.030 mg/kg, 0.050 mg/kg, 0.075 mg/kg, 0.100 mg/kg, or 0.134 mg/kg.

6. The anti-EFNA4 antibody-drug conjugate for use of any one of claims 1 to 5, wherein the anti-EFNA4 antibody-drug conjugate is administrable weekly (QW) at a dose of about 0.010 mg/kg, 0.015 mg/kg or 0.020 mg/kg.

7. The anti-EFNA4 antibody-drug conjugate for use of claim 6, wherein the anti-EFNA4 antibody-drug conjugate is administrable weekly (QW) at a dose of about 0.015 mg/kg.

8. The anti-EFNA4 antibody-drug conjugate for use of any one of claims 1 to 5, wherein the anti-EFNA4 antibody-drug conjugate is administrable every 3 weeks (Q3W) at a dose of about 0.015 mg/kg, 0.030 mg/kg, 0.050 mg/kg, 0.075 mg/kg, 0.100 mg/kg, or 0.134 mg/kg.

9. The anti-EFNA4 antibody-drug conjugate for use of any one of claims 1 to 8, wherein the anti-EFNA4 antibody-drug conjugate is administrable intravenously, subcutaneously, intramuscularly, by bolus injection, intracerebrally or by sustained release.

10. The anti-EFNA4 antibody-drug conjugate for use of any one of claims 1 to 9, wherein the anti-EFNA4 antibody-drug conjugate is formulated in a pharmaceutical composition.

11. The anti-EFNA4 antibody-drug conjugate for use of any one of claims 1 to 10, wherein the anti-EFNA4 antibody-drug conjugate comprises an antibody having three CDRS from a heavy chain variable region (VH) having the amino acid sequence shown in SEQ ID NO: 1 and three CDRS from a light chain variable region (VL) having the amino acid sequence shown in SEQ ID NO: 8.

12. The anti-EFNA4 antibody-drug conjugate for use of any one of claims 1 to 11, wherein the antibody comprises a VH CDR1 having the amino acid sequence shown in SEQ ID NO: 2, VH CDR2 having the amino acid sequence shown in SEQ ID NO: 4, and VH CDR3 having the amino acid sequence shown in SEQ ID NO: 6, and/or VL CDR1 having the amino acid sequence shown in SEQ ID NO: 9, VL CDR2 having the amino acid sequence shown in SEQ ID NO: 11, and VL CDR3 having the amino acid sequence shown in SEQ ID NO: 12.

13. The anti-EFNA4 antibody-drug conjugate for use of any one of claims 1 to 12, wherein the antibody is huE22.

14. The anti-EFNA4 antibody-drug conjugate for use of any one of claims 1 to 13, wherein the anti-EFNA4 antibody-drug conjugate further comprises a drug and a linker.

15. The anti-EFNA4 antibody-drug conjugate for use of any one of claims 1 to 14, wherein the drug is calicheamicin or a calicheamicin derivative (CM) and the linker is 4-(4′acetylphenoxy)butanoic acid (AcBut).

16. The anti-EFNA4 antibody-drug conjugate for use of any one of claims 1 to 15, wherein the anti-EFNA4 antibody-drug conjugate is huE22-AcBut-CM.

17. The anti-EFNA4 antibody-drug conjugate for use of any one of claims 1 to 16, wherein the cancer and/or EFNA4-associated disorder is characterized by overexpression of EFNA4.

18. The anti-EFNA4 antibody-drug conjugate for use of any one of claims 1 to 17, wherein the cancer or EFNA4-associated disorder is breast cancer, triple-negative breast cancer (TNBC), ovarian cancer, colorectal cancer, liver cancer, hepatocellular carcinoma (HCC), lung cancer non-small cell lung cancer (NSCLC), small cell lung cancer (SCLC), appendiceal cancer, spindle cell sarcoma, pancreatic cancer, cervical adenocarcinoma, peritoneal cancer, metastatic melanoma, renal cancer, neoplasm peritoneum, leukemia or chronic lymphocytic leukemia (CLL).

19. The anti-EFNA4 antibody-drug conjugate for use of claim 18, wherein the cancer is triple-negative breast cancer (TNBC).

20. The anti-EFNA4 antibody-drug conjugate for use of claim 18, wherein the cancer is ovarian cancer.

21. The anti-EFNA4 antibody-drug conjugate for use of any one of claims 1 to 20, wherein the treatment results in a decrease in tumor size of at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% as compared to the tumor size in the patient prior to first administration of the anti-EFNA4 antibody-drug conjugate.

22. The anti-EFNA4 antibody-drug conjugate for use of any one of claims 1 to 21, wherein the treatment is an initial treatment.

23. The anti-EFNA4 antibody-drug conjugate for use of any one of claims 1 to 21, wherein the cancer and/or EFNA4-associated disorder is unresponsive to conventional therapies.

24. The anti-EFNA4 antibody-drug conjugate for use of any one of claims 1 to 23, wherein the patient is a human.

25. A method for the treatment of a patient having cancer and/or an EFNA4-associated disorder, comprising administering to the patient an effective amount of an anti-EFNA4 antibody-drug conjugate (ADC) at least twice every week, at least weekly (QW), at least every 2 weeks (Q2W), at least every 3 weeks (Q3W) or at least every 4 weeks (Q4W).

26. The method of claim 25, wherein the anti-EFNA4 antibody-drug conjugate is administered weekly (QW) or every 3 weeks (Q3W).

27. The method of claim 25 or 26, wherein the anti-EFNA4 antibody-drug conjugate is administered at a dose of about 0.010 mg/kg to about 10 mg/kg, about 0.010 mg/kg to about 5 mg/kg, about 0.010 mg/kg to about 1 mg/kg or about 0.010 mg/kg to about 0.500 mg/kg.

28. The method of any one of claims 25 to 27, wherein the anti-EFNA4 antibody-drug conjugate is administered at a dose of at least 0.010, 0.015, 0.020, 0.025, 0.030, 0.035, 0.040, 0.045, 0.050, 0.055, 0.060, 0.065, 0.070, 0.075, 0.080, 0.095, 0.100, 0.150, 0.200, 0.250, 0.300, 0.350, 0.400, 0.450 or 0.500 mg/kg.

29. The method of any one of claims 25 to 28, wherein the anti-EFNA4 antibody-drug conjugate is administered at a dose of about 0.010 mg/kg, 0.015 mg/kg, 0.02 mg/kg, 0.030 mg/kg, 0.050 mg/kg, 0.075 mg/kg, 0.100 mg/kg, or 0.134 mg/kg.

30. The method of any one of claims 25 to 29, wherein the anti-EFNA4 antibody-drug conjugate is administered weekly (QW) at a dose of about 0.010 mg/kg, 0.015 mg/kg or 0.020 mg/kg.

31. The method of claim 30, wherein the anti-EFNA4 antibody-drug conjugate is administered weekly (QW) at a dose of about 0.015 mg/kg.

32. The method of any one of claims 25 to 29, wherein the anti-EFNA4 antibody-drug conjugate is administered every 3 weeks (Q3W) at a dose of about 0.015 mg/kg, 0.030 mg/kg, 0.050 mg/kg, 0.075 mg/kg, 0.100 mg/kg, or 0.134 mg/kg.

33. The method of any one of claims 25 to 32, wherein the anti-EFNA4 antibody-drug conjugate is administered intravenously, subcutaneously, intramuscularly, by bolus injection, intracerebrally or by sustained release.

34. The method of any one of claims 25 to 33, wherein the anti-EFNA4 antibody-drug conjugate is formulated in a pharmaceutical composition.

35. The method of any one of claims 25 to 34, wherein the anti-EFNA4 antibody-drug conjugate comprises an antibody having three CDRS from a heavy chain variable region (VH) having the amino acid sequence shown in SEQ ID NO: 1 and three CDRS from a light chain variable region (VL) having the amino acid sequence shown in SEQ ID NO: 8.

36. The method of any one of claims 25 to 35, wherein the antibody comprises a VH CDR1 having the amino acid sequence shown in SEQ ID NO: 2, VH CDR2 having the amino acid sequence shown in SEQ ID NO: 4, and VH CDR3 having the amino acid sequence shown in SEQ ID NO: 6, and/or VL CDR1 having the amino acid sequence shown in SEQ ID NO: 9, VL CDR2 having the amino acid sequence shown in SEQ ID NO: 11, and VL CDR3 having the amino acid sequence shown in SEQ ID NO: 12.

37. The method any one of claims 25 to 36, wherein the antibody is huE22.

38. The method of any one of claims 25 to 37, wherein the anti-EFNA4 antibody-drug conjugate further comprises a drug and a linker.

39. The method of any one of claims 25 to 38, wherein the drug is calicheamicin or a calicheamicin derivative (CM) and the linker is 4-(4′acetylphenoxy)butanoic acid (AcBut).

40. The method any one of claims 25 to 39, wherein the anti-EFNA4 antibody-drug conjugate is huE22-AcBut-CM.

41. The method of any one of claims 25 to 40, wherein the cancer and/or EFNA4-associated disorder is characterized by overexpression of EFNA4.

42. The method of any one of claims 25 to 41, wherein the cancer or EFNA4-associated disorder is breast cancer, triple-negative breast cancer (TNBC), ovarian cancer, colorectal cancer, liver cancer, hepatocellular carcinoma (HCC), lung cancer non-small cell lung cancer (NSCLC), small cell lung cancer (SCLC), appendiceal cancer, spindle cell sarcoma, pancreatic cancer, cervical adenocarcinoma, peritoneal cancer, metastatic melanoma, renal cancer, neoplasm peritoneum, leukemia or chronic lymphocytic leukemia (CLL).

43. The method of claim 42, wherein the cancer is triple negative breast cancer (TNBC).

44. The method of claim 42, wherein the cancer is ovarian cancer.

45. The method of any one of claims 25 to 44, wherein the treatment results in a decrease in tumor size of at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% as compared to the tumor size in the patient prior to first administration of the anti-EFNA4 antibody-drug conjugate.

46. The method of any one of claims 25 to 45, wherein the treatment is an initial treatment.

47. The method of any one of claims 25 to 45, wherein the cancer and/or EFNA4-associated disorder is unresponsive to conventional therapies.

48. The method of any one of claims 25 to 47, wherein the patient is a human.
